(19) United States
(12) Patent Application Publication
(10) Pub. No.: US 2025/0311987 A1
(43) Pub. Date: Oct. 9, 2025

(54) X-RAY COMPUTED TOMOGRAPHY APPARATUS

(71) Applicant: Canon Medical Systems Corporation, Otawara-shi (JP)

(72) Inventor: Ryusei SAIKI, Nasushiobara (JP)

(73) Assignee: Canon Medical Systems Corporation, Otawara-shi (JP)

(21) Appl. No.: 19/096,817

(22) Filed: Apr. 1, 2025

(30) Foreign Application Priority Data
Apr. 5, 2024 (JP) 2024-061163

(51) Int. Cl. A61B 6/03; A61B 6/00; A61B 6/12; A61B 6/46

(52) U.S. Cl. CPC A61B 6/032; A61B 6/12; A61B 6/4447; A61B 6/461

(57) ABSTRACT

An X-ray computed tomography apparatus according to an embodiment includes a gantry body and processor. The gantry body supports an X-ray tube, an X-ray detector, and a data acquisition unit to be rotatable about a central axis of a bore. The X-ray tube generates X-rays, the X-ray detector detects X-rays generated by the X-ray tube and passing through a subject, and the data acquisition circuitry acquires projection data via the X-ray detector. The processor corrects detection angle information of the projection data in accordance with a correction amount based on a first reference angle in a rotation direction of the X-ray tube and a subject angle determining an orientation of the subject in the bore. The processor reconstructs a CT image relating to the subject based on the projection data and the corrected detection angle information.

### CROSS-REFERENCE TO RELATED APPLICATIONS

This application is based upon and claims the benefit of priority from Japanese Patent Application No. 2024-061163, filed Apr. 5, 2024, the entire contents of which are incorporated herein by reference.

### FIELD

Embodiments described herein relate generally to an X-ray computed tomography apparatus.

### BACKGROUND

An X-ray computed tomography apparatus reconstructs a CT image based on projection data acquired in association with angle information of a rotation direction of an X-ray tube. Basically, the CT image is reconstructed in such a manner that a rotation frame 0° position in a gantry comes to an upper part of the image.

There is also a type of X-ray computed tomography apparatus capable of performing upright imaging and seated imaging, allowing imaging in any orientation with respect to the gantry. For the upright imaging and the seated imaging, because of the reconstruction being performed based on angle information, there is a case where an orientation of the subject at the time of imaging and an orientation of the subject on the image may not coincide with each other. In this case, if image processing for rotating the reconstructed image is performed to match the orientation of the subject on the image with the orientation of the subject at the time of imaging, blurring of the image may occur or the time required for image processing may be extended depending on the angle of rotation.

### BRIEF DESCRIPTION OF THE DRAWINGS

FIG. 1 is a diagram showing a configuration example of an X-ray computed tomography apparatus according to a first embodiment.

FIG. 2 is a view illustrating an imaging posture of the X-ray computed tomography apparatus according to the first embodiment.

FIG. 3 is another view illustrating the imaging posture of the X-ray computed tomography apparatus according to the first embodiment.

FIG. 4 is a view showing a relation between a positional relation between the gantry body and the subject and an image to be reconstructed.

FIG. 5 schematically illustrates a flow of a CT examination according to the first embodiment.

FIG. 6 is a view illustrating determination of a subject angle.

FIG. 7 is a view illustrating calculation of a correction amount.

FIG. 8 is a diagram illustrating correction of detection angle information.

FIG. 9 is a diagram illustrating CT images to be reconstructed from uncorrected projection data and corrected projection data.

FIG. 10 is a view illustrating orientations of a subject in CT imaging in a decubitus position and an upright position.

FIG. 11 is a view illustrating a display screen.

FIG. 12 is a view showing a configuration example of an X- ray computed tomography apparatus according to a second embodiment.

FIG. 13 schematically illustrates a flow of a CT examination according to the second embodiment.

FIG. 14 is a diagram illustrating a correction amount stored in association with projection data.

FIG. 15 is a view showing a configuration example of an X-ray computed tomography apparatus according to a third embodiment.

FIG. 16 is a view showing an orientation of a subject and an exposure start position in decubitus imaging.

FIG. 17 is a view showing an orientation of a subject and a position of an X-ray tube in upright imaging.

### DETAILED DESCRIPTION

An X-ray computed tomography apparatus according to an embodiment includes a gantry body, a correction unit, and a reconstruction unit. The gantry body supports an X-ray tube, an X-ray detector, and a data acquisition unit to be rotatable about a central axis of a bore. The X-ray tube generates X-rays, the X-ray detector detects X-rays that have been generated by the X-ray tube and passed through a subject, and the data acquisition circuitry acquires projection data via the X-ray detector. The correction unit corrects detection angle information of the projection data in accordance with a correction amount that is based on a first reference angle in the rotation direction of the X-ray tube and a subject angle determining an orientation of the subject in the bore. The reconstruction unit reconstructs a CT image relating to the subject based on the projection data and the corrected detection angle information.

### First Embodiment

Hereinafter, embodiments of the X-ray computed tomography apparatus will be described in detail with reference to the accompanying drawings.

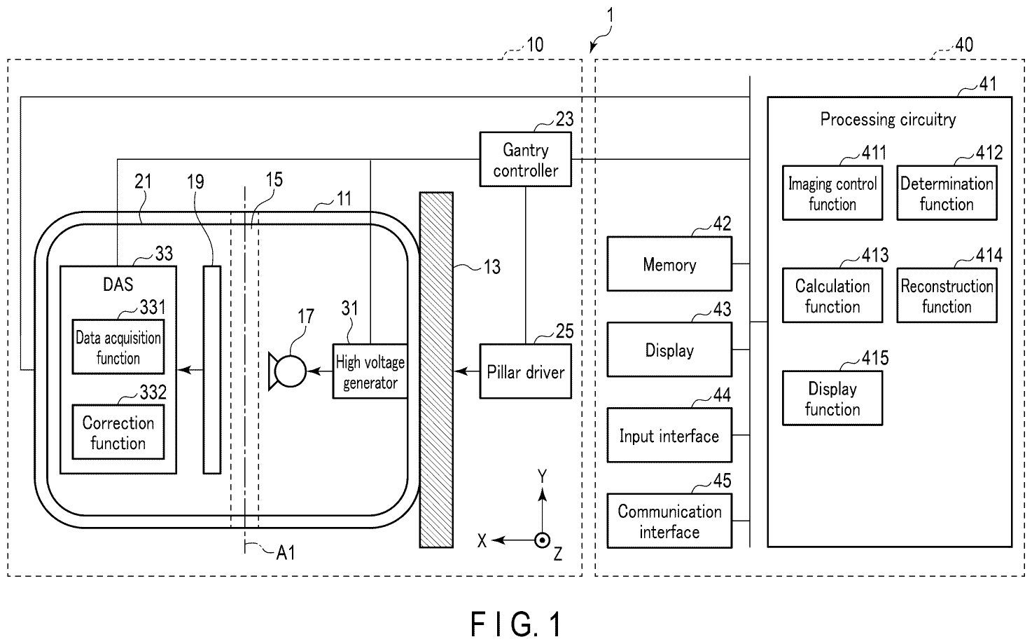

FIG. 1 is a diagram showing a configuration of an X-ray computed tomography apparatus 1 according to a first embodiment. The X-ray computed tomography apparatus 1 applies X-rays to a subject from an X-ray tube 17 and detects the applied X-rays with an X-ray detector 19. The X-ray computed tomography apparatus 1 generates CT images relating to the subject based on an output from the X-ray detector 19.

As shown in FIG. 1, the X-ray computed tomography apparatus 1 includes a gantry 10 and a console 40. For example, the gantry 10 is installed in a CT examination room, and the console 40 is installed in a control room adjacent to the CT examination room. The gantry 10 and the console 40 are connected to each other in a wired or wireless manner. The gantry 10 is equipped with a mechanism for applying X-ray computed tomography (hereinafter “X-ray CT imaging”) to a subject in a decubitus position or an upright position. The console 40 is a computer that controls the gantry 10. The X-ray computed tomography apparatus 1 is also applicable to a seated position instead of the upright position.

Although illustration is omitted in FIG. 1, the X-ray computed tomography apparatus 1 further includes a table device on which a subject is placed. The table device may be included in the X-ray computed tomography apparatus 1 or may be outside the X-ray computed tomography apparatus 1. The X-ray computed tomography apparatus 1 may include a support device that supports the subject in upright imaging. The support device corresponds to a table top of the table device in the decubitus CT. The support device may be fixed to a floor surface or movable while supporting the subject. The support device may serve as a table device. The X-ray computed tomography apparatus 1 may not include a support device used for upright imaging.

A direction perpendicular to the floor surface will be referred to as a Y-axis direction, a direction horizontally orthogonal to the Y-axis direction and a rotation axis direction of the gantry body 11 during decubitus imaging will be referred to as a Z-axis direction, and a direction horizontally orthogonal to the Y-axis direction and the Z-axis direction will be referred to as an X-axis direction. Furthermore, a-Y axis direction will be referred as a lower side, a +Y axis direction will be referred as an upper side, a −X axis direction will be referred as a rear side, and a +X axis direction will be referred as a front side. The Y axis is parallel to a central axis A1 of the gantry body 11 during upright imaging. For example, in a case of upright imaging, the subject enters the lower side of the gantry body 11 from the rear side.

As shown in FIG. 1, the gantry 10 includes the gantry body 11 and a pillar 13. The gantry body 11 performs X-ray CT imaging. The gantry body 11 is an approximately cylindrical structure in which an opening (bore) 15 is formed. The gantry body 11 accommodates the X-ray tube 17 and the X-ray detector 19 arranged to face each other with the bore 15 interposed therebetween, a high voltage generator 31, and data acquisition circuitry (DAS: Data Acquisition System) 33.

More specifically, the gantry body 11 further includes a main frame (not shown) made of a metal such as aluminum, and a rotation frame 21 supported to be rotatable via a bearing or the like about the central axis A1 by the main frame. In the main frame, a contact portion of the rotation frame 21 is provided with an annular electrode (not shown). A conductive slider (not shown) is attached to the contact portion of the main frame so as to come into sliding contact with the annular electrode. The rotation frame 21 is a metal frame made of a metal such as aluminum and formed in an annular shape, and for example, the X-ray tube 17 and the X-ray detector 19 are attached thereto.

Upon receiving power from a rotation driver (not shown), the rotation frame 21 rotates about the central axis A1 of the bore 15 at a constant angle speed. The rotation driver produces power for rotating the rotation frame 21 under the control of a gantry controller 23. The rotation driver is realized by, for example, a motor such as a direct driver motor, a servomotor, or the like.

The pillar 13 is a base that supports the gantry body 11 away from the floor surface. The pillar 13 has, for example, a column shape such as a cylinder shape or a prismatic shape. The pillar 13 is attached to, for example, a side surface portion of the gantry body 11. The pillar 13 supports the gantry body 11 to be slidable in the perpendicular direction with respect to the floor surface while the central axis A1 of the bore 15 maintains the perpendicular direction with respect to the floor surface so as to perform X-ray CT imaging on the subject in an upright posture or a seated posture.

Typically, the pillar 13 is provided on one side portion of the gantry body 11. However, the present embodiment is not limited to this. For example, two pillars 13 may be connected to both side portions of the gantry body 11. That is, at least one pillar 13 supports the gantry body 11 so as to be movable in the vertical direction. The pillar 13 has a column-like shape, but the present embodiment is not limited thereto. For example, the pillar 13 may have any shape such as a U shape as long as the pillar 13 can support at least one side portion of the gantry body 11.

As shown in FIG. 1, the pillar 13 accommodates a driver (hereinafter “pillar driver”) 25 for sliding the gantry body 11 in the perpendicular direction. The pillar driver 25 produces power for sliding the gantry body 11 in the perpendicular direction under the control of the gantry controller 23. For example, the pillar driver 25 produces power through driving at a rotation speed corresponding to a duty cycle or the like of a drive signal from the gantry controller 23. Upon receiving power from the pillar driver 25, the pillar 13 slides the gantry body 11 in the perpendicular direction with respect to the pillar 13. The pillar driver 25 is realized by a motor, for example a servomotor or the like.

The pillar 13 supports the gantry body 11 to be rotatable about a horizontal axis X. Specifically, upon receiving power from the pillar driver 25, the pillar 13 rotates the gantry body 11 about the horizontal axis with respect to the pillar 13. The pillar driver 25 rotates the gantry body 11 between the horizontal direction and the vertical direction through rotation of an internal gear in a gyratory bearing, for example under the control of the gantry controller 23. The rotation mechanism for rotating the gantry body 11 is not limited to the gyratory bearing and may be realized by known mechanisms. The rotation of the gantry body 11 by the rotation mechanism allows switching between upright imaging and decubitus imaging.

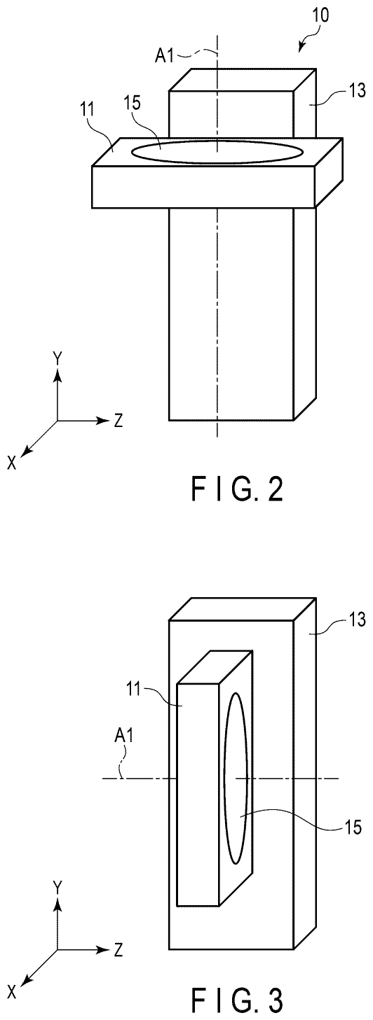

FIG. 2 shows a posture of the gantry 10 for upright imaging, and FIG. 3 shows a posture of the gantry 10 for decubitus imaging. The gantry body 11 shown in FIG. 2 is supported in such a manner that the bore 15 is directed toward the perpendicular direction with respect to the floor surface. The gantry body 11 shown in FIG. 3 is supported in such a manner that the bore 15 is directed toward the horizontal direction with respect to the floor surface. The pillar 13 shown in FIGS. 2 and 3 supports the gantry body 11 to be rotatable about the horizontal axis in such a manner that the bore 15 is directed toward the horizontal direction or the perpendicular direction with respect to the floor surface.

As shown in FIG. 1, the X-ray tube 17 is supplied with a high voltage from the high voltage generator 31 and generates X-rays. The high voltage generator 31 is attached to, for example, the rotation frame 21. The high voltage generator 31 generates a high voltage to be applied to the X-ray tube 17 under the control of the gantry controller 23 from the power supplied via the annular electrode from a power supply (not shown) of the gantry body 11. The high voltage generator 31 and the X-ray tube 17 are connected via a high voltage cable (not shown). A high voltage generated by the high voltage generator 31 is applied to the X-ray tube 17 via the high voltage cable.

The X-ray detector 19 detects X-rays that have been generated by the X-ray tube 17 and passed through the subject. The X-ray detector 19 is equipped with a plurality of X-ray detection elements (not shown) arranged in a two-dimensional curved surface. Each X-ray detection element detects an X-ray from the X-ray tube 17 and converts the detected X-ray into an electric signal having a peak value corresponding to an intensity of the detected X-ray. Each X-ray detection element has, for example, a scintillator and a photoelectric conversion element. The scintillator generates fluorescence upon receiving the X-ray. The photoelectric conversion element coverts the generated fluorescence into a charge pulse. The charge pulse has a peak value corresponding to the intensity of the X-ray. As the photoelectric conversion element, specifically, a circuit element that converts fluorescence into an electrical signal, such as a photomultiplier tube or a photodiode, is used. The X-ray detector 19 according to the present embodiment is not limited to an indirect conversion type detector that converts X-rays into fluorescence and then into electrical signals, and may be a direct conversion type detector that directly converts X-rays into electrical signals.

The DAS 33 is realized by, for example, a processor such as a semiconductor integrated circuit in which integration circuitry and an A/D converter provided for each of the plurality of X-ray detection elements are arranged in parallel. The DAS 33 executes a data acquisition function 331 and a correction function 332 with a processor that executes the program loaded into the memory. The functions 331 and 332 are not limited to those implemented by single processing circuitry. Semiconductor integrated circuitry may be configured by combining a plurality of independent processors that execute respective programs to implement the respective functions 331 and 332.

Through implementation of the data acquisition function 331, the DAS 33 acquires, for each view, digital data indicating the intensity of X-rays attenuated by the subject. The DAS 33 is connected to, for example, the X-ray detector 19 in the gantry body 11. The integration circuitry integrates the electric signals from the X-ray detection elements over a predetermined view period and generates integral signals. The A/D converter performs A/D conversion on the generated integral signals and generates digital data having data values corresponding to peak values of the integral signals. The converted digital data is referred to as projection data. The projection data is a set of digital values of X-ray doses identified through a channel number and a row number of the X-ray detection element of the generation source, and a view number indicating the acquired view. The projection data is supplied, for example, to the console 40 via a non-contact data transmission device (not shown) in the gantry body 11.

The DAS 33 corrects, through implementation of the correction function 332, detection angle information of the projection data in accordance with a correction amount that is based on a first reference angle and a subject angle. The first reference angle is an angle serving as a reference for calculating the correction amount in the rotation direction of the X-ray tube. The first reference angle is set to an image reconstruction start angle. The subject angle is an angle that determines an orientation of the subject placed in the bore 15 in the rotational direction of the X-ray tube. The detection angle information indicates at which angle the X-rays detected by the X-ray detector 19 are generated in the rotation direction of the X-ray tube 17. For example, the detection angle information may be associated with a view number and/or a digital value of the X-ray dose as projection data.

The gantry controller 23 controls the pillar driver 25, the high voltage generator 31, the DAS 33 and the like in accordance with commands from the console 40. The gantry controller 23 includes, as hardware resources, a processor such as a central processing unit (CPU) and a storage device (memory) such as a read only memory (ROM) and a random access memory (RAM). The gantry driving system is a driving system relating to components of the gantry body 11 such as the high voltage generator 31, the pillar driver 25, and the rotation driver of the rotation frame 21.

The console 40 includes processing circuitry 41, a memory 42, a display 43, an input interface 44, and a communication interface 45. The processing circuitry 41, the memory 42, the display 43, the input interface 44, and the communication interface 45 perform data communications with each other via a bus. While the console 40 is described as being independent of the gantry 10, the console 40 or some components of the console 40 may be included in the gantry 10.

The memory 42 is a storage device configured to store various types of information such as a hard disk drive (HDD), a solid state drive (SSD), and integrated circuitry. The memory 42 stores, for example, the projection data and the reconstructed image data. In addition to an HDD and SSD, the memory 42 may be a portable storage medium such as a compact disc (CD), a digital versatile disc (DVD), and a flash memory. The memory 42 may be a driver that allows for reading and writing of information of various types from and to a semiconductor memory element such as a flash memory and a random access memory (RAM). The storage region of the memory 42 may be provided in the X-ray computed tomography apparatus 1, or in an external storage device connected thereto by way of a network. The memory 42 stores databases described later.

The display 43 is configured to display various types of information. For example, the display 43 outputs a medical image (CT image) generated by the processing circuitry 41, a graphical user interface (GUI) for receiving various operations from the user, and the like. For the display 43, a variety of displays may be used as appropriate. For example, as the display 43, a liquid crystal display (LCD), a cathode ray tube (CRT) display, an organic electro luminescence display (OELD), or a plasma display can be used as appropriate. The display 42 may be provided in the gantry 10. Alternatively, the display 43 may be a desktop type, or may be constituted by a tablet terminal or the like that can wirelessly communicate with the main body of the console 40.

The input interface 44 receives various input operations from the user, converts the received input operations into electrical signals, and outputs them to the processing circuitry 41. For example, the input interface 44 receives, from the user, an acquisition condition in acquiring projection data, a subject angle indicating an orientation of the subject, and the like. As the input interface 44, for example, a mouse, a keyboard, a trackball, a switch, a button, a joystick, a touch pad, a touch panel display, and the like can be used as appropriate. In the present embodiment, the input interface 44 is not limited to one including physical operation components such as a mouse, a keyboard, a trackball, a switch, a button, a joystick, a touch pad, and a touch panel display. For instance, examples of the input interface 44 may include an electric signal processing circuit configured to receive an electric signal corresponding to an input operation from an external input device provided separately from the apparatus, and output this electric signal to the processing circuitry 41. The input interface 44 may be provided in the gantry 10. Alternatively, the input interface 44 may be constituted by a tablet terminal or the like that can wirelessly communicate with the main body of the console 40.

The communication interface 45 is an interface for performing data communication with other computers. For instance, the communication interface 45 transmits and receives the projection data and/or the CT image data to and from a picture archiving and communication system (PACS) via a network.

The processing circuitry 41 controls the entire operation of the X-ray computed tomography apparatus 1 in accordance with the electrical signal of the input operation output from the input interface 44. For instance, the processing circuitry 41 includes a processor such as a CPU and a memory such as a ROM and a RAM as hardware resources. The processing circuitry 41 implements an imaging control function 411, a determination function 412, a calculation function 413, a reconstruction function 414, a display control function 415 and the like, with a processor that executes the program loaded into the memory. The functions 411 to 415 are each not necessarily implemented by single processing circuitry. Processing circuitry may be configured by combining a plurality of independent processors, and the processors may execute respective programs to implement the functions 411 to 415.

As the imaging control function 411, the processing circuitry 41 issues a command to the gantry controller 23 so that the gantry 10 performs X-ray CT imaging according to scan conditions. The gantry controller 23 controls the pillar driver 25, the high voltage generator 31, the DAS 33 and the like so as to perform X-ray CT imaging in accordance with a command from the console 40.

As the determination function 412, the processing circuitry 41 determines a subject angle representing an orientation of the subject. As means for determination, for example, the input interface 44 may be used, the projection data may be used, an optical camera may be used, or various physical sensors may be used.

As the calculation function 413, the processing circuitry 41 calculates a correction amount to be used by the correction function 332 of the DAS 33 based on the subject angle and a second reference angle. The second reference angle is a reference angle of the rotating reference frame of the CT image. For example, the second reference angle is an angle at which the rotation angle about the center point of the CT image is positioned at approximately the center of the upper side. The second reference angle can be freely determined. The correction amount is an amount of change of the detection angle information.

As the reconstruction function 414, the processing circuitry 41 reconstructs the CT image relating to the subject based on the projection data output from the DAS 33 and the detection angle information. The processing circuitry 41 reconstructs the CT image in such a manner that the direction of the subject angle is directed toward approximately the center of the upper side. In a case where the detection angle information is corrected with the correction function 332, the processing circuitry 41 reconstructs the CT image relating to the subject based on the projection data and the detection angle information corrected with the correction function 332. The CT image represents a spatial distribution of CT values evaluating an attenuation coefficient of a substance. The processing circuitry 41 converts the CT image into a cross-sectional image of a given cross section or a rendering image in a given direction of a viewpoint. The conversion is performed based on an input operation received from the user via the input interface 43. For example, the processing circuitry 41 performs three-dimensional image processing, such as volume rendering, surface volume rendering, image value projection processing, multi-planer reconstruction (MPR) processing, or curved MPR (CPR) processing, on the CT image, thereby generating rendering image data in the given direction of a viewpoint. As an image reconstruction algorithm, an existing image reconstruction algorithm such as a filtered back projection (FBP) method or a successive approximation reconstruction method may be used.

As the display control function 415, the processing circuitry 41 displays various information related to CT imaging on the display 43.

An operation example of the X-ray computed tomography apparatus according to the first embodiment will be described below.

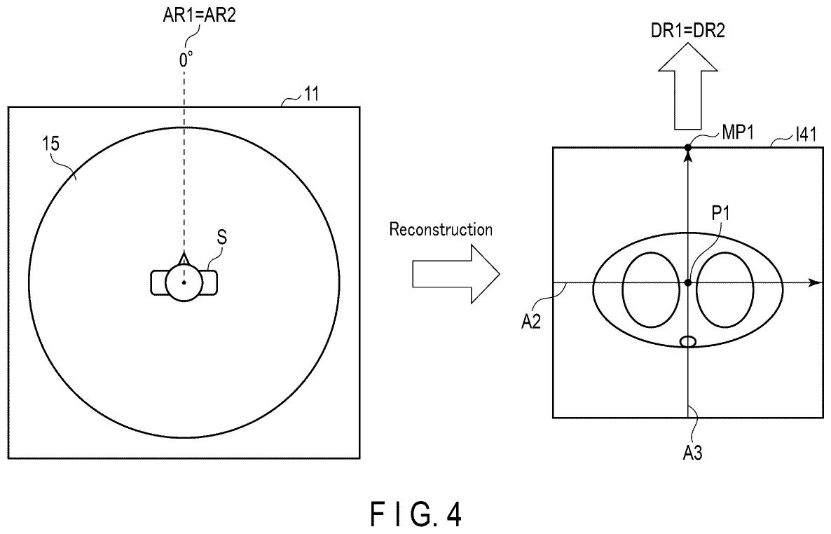

FIG. 4 shows the first reference angle, the second reference angle, and the orientation of the CT image. A first reference angle AR1 is an angle serving as a reference of an angle along the rotation direction of the X-ray tube 17. In the present embodiment, the first reference angle AR1 is 0°. A CT image I41 is defined by a Cartesian coordinate system formed by a horizontal axis A2 and a vertical axis A3 orthogonal to each other at a center point P1, and a rotating reference frame determining a deviation angle clockwise from the vertical axis A3. A second reference angle AR2 is a reference angle of the rotating reference frame of the CT image. The first reference angle AR1 is set to coincide with the second reference angle AR2. As an example, the second reference angle AR2 is set to an angle at which the rotation angle about the center point of the CT image is positioned at approximately the center of the upper side. In this case, as shown in FIG. 4, the second reference angle AR2 is 0°. That is, rotation angle 0° of the X-ray tube 17 is set to be positioned at 0° of the coordinate system of the image space. A direction DR2 of the second reference angle AR2 coincides with a positive direction of the vertical axis A3. In other words, in the rotating reference frame, the direction DR2 is the direction of 0°. The direction DR2 is the same direction as a direction DR1 of the first reference angle. In the CT image I41, the front of the image of the subject S is the direction DR2. This is because the front of the subject S in the bore 15 is directed toward the direction DR2 during CT imaging. As another example, in a case where the left side of the subject S in the bore 15 is directed toward the direction DR2 during CT imaging, a CT image in which the image of the subject S in the CT image I41 is rotated by −90° in the rotating reference frame is obtained.

The first reference angle and the second reference angle may not coincide with each other, but hereinafter it is assumed for concrete description that the first reference angle and the second reference angle coincide with each other at 0° in the rotation direction of the X-ray tube 17.

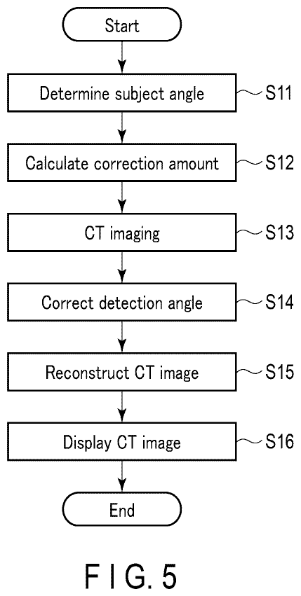

FIG. 5 shows a processing procedure of a CT examination according to the first embodiment.

As shown in FIG. 5, the processing circuitry 41 determines a subject angle through implementation of the determination function 412 (step S11). The subject in step S11 is assumed to not be positioned in the bore 15. The subject angle represents an angle that determines an orientation of the subject on the setting. The subject is expected to be actually placed in the bore 15 to be positioned at the subject angle determined in step S11.

The subject angle is an angle that determines an orientation of the subject placed in the bore 15 in the rotational direction of the X-ray tube. The subject angle may be defined as any body orientation of the subject, an angle in a lateral direction of the subject, or an angle in a back direction of the subject. The subject angle may be defined as, for example, a front direction of the subject.

The subject angle may be determined to be any angle. The method of determining the subject angle is not particularly limited. For example, the processing circuitry 41 determines the subject angle in accordance with an instruction from the user, such as a method of directly inputting a numerical value or a method of determining the subject angle using a GUI.

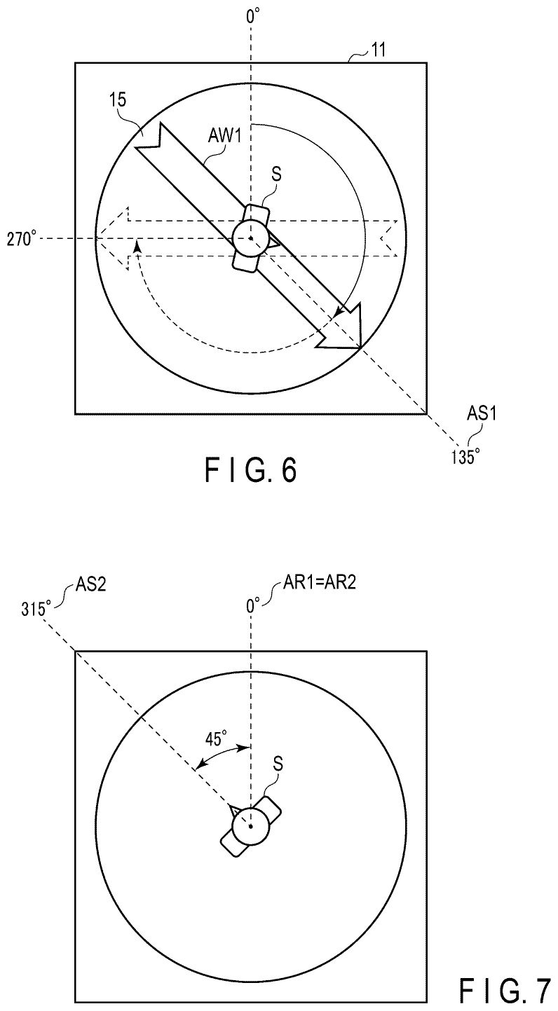

FIG. 6 shows the subject angle. An arrow AW1 shown in FIG. 6 is a GUI component that indicates a subject angle AS1. As shown in FIG. 6, the subject angle AS1 is determined in accordance with an instruction from the user via the GUI. For example, by moving the arrow AW1 about the central axis A1 of the bore 15 in accordance with the instruction from the user, the subject angle AS1 is determined according to the angle of the arrow AW1. By using the GUI, it is possible to intuitively determine the subject angle.

Upon performing step S11, the processing circuitry 41 calculates, through implementation of the calculation function 413, a correction amount in such a manner that the detection angle information corresponding to the subject angle determined in step S11 substantially coincides with the second reference angle (step S12). The processing circuitry 41 transmits the calculated correction amount to the DAS 33 of the gantry body 11 via the bus.

FIG. 7 shows an example of calculating the correction amount. As shown in FIG. 7, it is assumed that the first reference angle AR1 is 0°, the subject angle AS2 is defined as the front of the subject S, and the subject angle AS2 is 315°. In this case, if no correction is performed, a CT image in which the front direction of the subject S is directed towards the direction shifted by +45° from the direction of approximately the center (0° direction) of the upper side of the CT image is obtained. In order to reconstruct a CT image in which the front of the subject S is directed toward the direction of the second reference angle AR2, it suffices that the projection data acquired at the front of the subject S is reconstructed as projection data acquired at the second reference angle AR2. That is, it suffices that the detection angle information of the projection data acquired at the subject angle AS2 is corrected to be the second reference angle AR2. Accordingly, the correction amount may be calculated by subtracting the subject angle from the second reference angle AR2 or an angle obtained by adding 360° to the second reference angle AS2. For example, the correction amount in FIG. 7 is calculated as −315° or +45°.

The correction amount may be calculated in such a manner that the second reference angle AR2 and the subject angle AS2 are any angles. For example, the correction may be made in such a manner that the second reference angle AR2 and the subject angle AS2 are 90°. In this case, the correction amount may be calculated by subtracting the subject angle from an angle obtained by adding 90° to the second reference angle AR2 or an angle obtained by adding 450° to the second reference angle AS2. Thus, a CT image in which the lateral direction of the subject S is directed toward the direction of approximately the center (0° direction) of the upper side of the CT image is obtained.

Upon performing step S12, the processing circuitry 41 performs CT imaging through implementation of the imaging control function 411 (step S13). Prior to CT imaging of S13, the subject is placed under the bore 15. Under the control of the imaging control function 411, the DAS 33 acquires projection data relating to the subject through implementation of the data acquisition function 331.

Upon performing step S13, the DAS 33 corrects, through implementation of the correction function 332, the detection angle information of the projection data acquired in step S13 in accordance with the correction amount calculated in step S12 at the time of acquiring the projection data (step S14). In the first embodiment, the DAS 33 corrects the detection angle information of the projection data. In other words, the detection angle information is corrected in the gantry body 11. After correction of the detection angle information, the non-contact data transmission device transmits the projection data to the console 40.

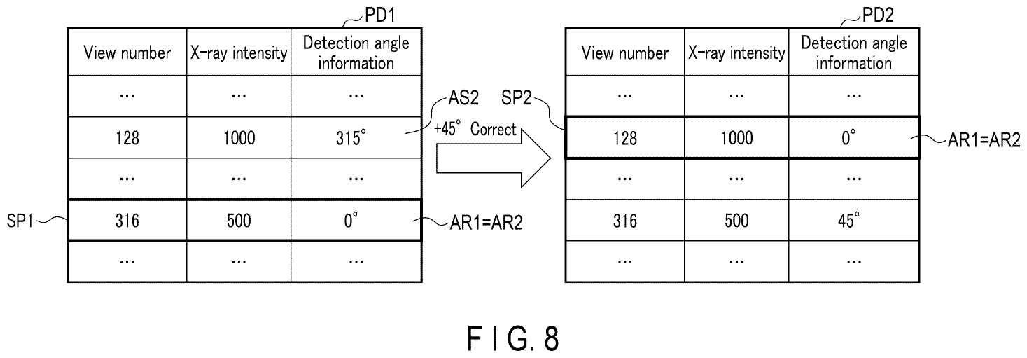

FIG. 8 shows uncorrected projection data PD1 and corrected projection data PD2 of the detection angle information. The uncorrected projection data PD1 and the corrected projection data PD2 each have a view number, an X-ray intensity, and detection angle information. The view number is a serial number of an X-ray sampling period. The X-ray intensity is an intensity of X-rays detected by the X-ray detector 19. The detection angle information indicates a rotation angle of the X-ray tube 17 during acquiring of the projection data.

In FIG. 8, the subject angle AS2 is assumed to be 315°. A reconstruction start position SP1 and a reconstruction start position SP2 are each an address position on the projection data at which reconstruction of a single CT image is started, and are each set to an address position of the projection data of the detection angle information of the first reference angle. The CT image is reconstructed in such a manner that the direction of the detection angle information of each of the reconstruction start positions SP1 and SP2 is directed toward approximately the center of the upper side. That is, if the first reference angle and the second reference angle coincide with each other, a CT image in which the direction of the second reference angle is directed toward approximately the center of the upper side is obtained. Therefore, by performing correction to shift the detection angle information in such a manner that the detection angle information of the subject angle AS2 becomes the first reference angle, a CT image in which the orientation of the subject angle is directed toward approximately the center of the upper side is obtained.

In FIG. 8, the reconstruction start positions SP1 and SP2 correspond to projection data in which the detection angle information is 0°. As an example, in a case where the correction amount is +45°, the processing circuitry 41 rewrites all of the detection angle information in the uncorrected projection data PD1 to an angle of +45°. For example, the detection angle information in the uncorrected projection data PD1 with a view number of 128, an X-ray intensity of 1000, and detection angle information of 315° is corrected to 0°. The correction of the detection angle information may be performed on the projection data acquired in all the channels and all the rows. Thus, the projection data corresponding to the detection angle information of the subject angle AS2 (the projection data of view number 128) can be arranged at the reconstruction start position SP2.

Upon performing step S14, the processing circuitry 41 reconstructs, through implementation of the reconstruction function 414, the CT image in such a manner that the subject angle coincides with the second reference angle in the CT image (step S15). In step S15, the processing circuitry 41 reconstructs the CT image based on the detection angle information corrected in step 14 and the projection data.

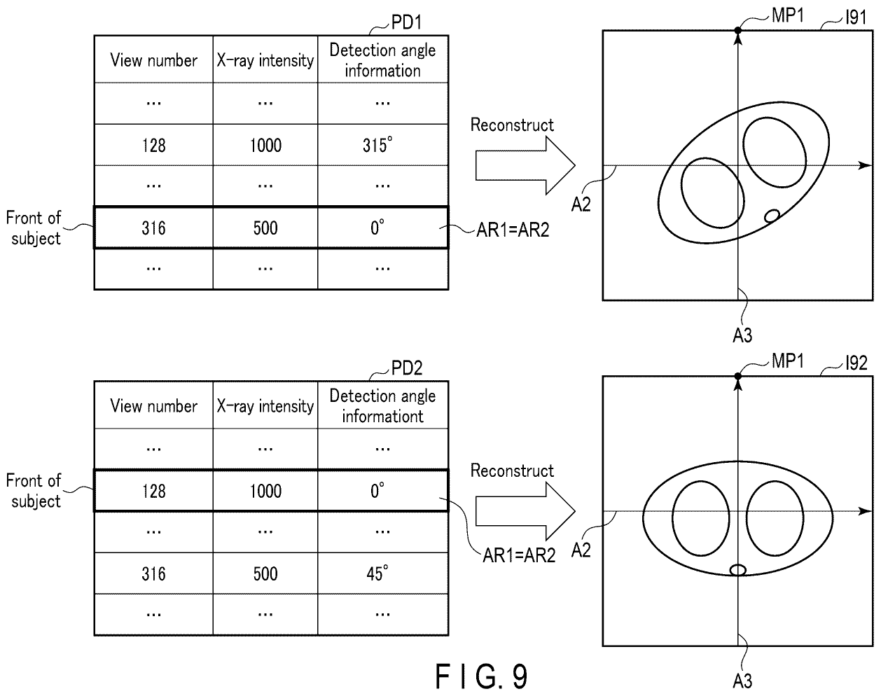

FIG. 9 shows the relation between the detection angle information and the orientation of the subject image in the CT image. The upper part of FIG. 9 shows the uncorrected projection data PD1 and a CT image I91 obtained by reconstructing the uncorrected projection data PD1. The lower part of FIG. 9 shows the corrected projection data PD2 and a CT image I92 obtained by reconstructing the corrected projection data PD2. The CT images I91 and I92 are generated by the same reconstruction processing. In the uncorrected projection data PD1, the detection angle information of the projection data corresponding to the front direction of the subject is 315°, which is different the angle of the second reference angle AR2, which is 0°. In this case, in the CT image I91, the front of the subject image is not directed toward the direction of approximately a center MP1 of the upper side. On the other hand, in the corrected projection data PD2, the detection angle information of the projection data corresponding to the front direction of the subject is 0°, which is the same angle as 0° of the second reference angle AR2, and therefore, in the CT image I92, the front of the subject image is directed toward the direction of approximately the center MP1 of the upper side. Thus, it is possible to reconstruct the CT image of the subject in which the direction of the subject angle is directed toward the upper side, regardless of the actual orientation of the subject, without changing the reconstruction processing.

Upon performing step S15, the processing circuitry 41 displays the CT image reconstructed in step S15 on the display 43 through implementation of the display control function 415 (step S16). For example, the processing circuitry 41 may display the CT image I92 of FIG. 9. It is possible to display the CT image of the subject in which the direction of the subject angle is directed toward a given direction regardless of the actual orientation of the subject. The processing circuitry 41 may display, for example, together with the CT image, the correction amount calculated in step S12, or an input screen for inputting the subject angle by the user.

Upon performing step S16, the CT examination according to the first embodiment ends.

The correction of the orientation of the subject on the CT image shown in FIG. 5 is an example, and various additions, changes, and/or deletions can be made without changing the gist of the first embodiment.

### First Modification

The processing circuitry 41 according to the first embodiment determines the subject angle with the GUI in accordance with an instruction from the user. Processing circuitry 41 according to the first modification determines the subject angle using a camera, a physical sensor, and/or projection data. In this case, since the subject angle is determined after the subject is positioned, the subject angle coincides with the orientation of the subject.

Specifically, the subject angle may be determined by imaging, by using a camera, a subject, a pole for fixing the posture of the subject in upright imaging, and/or a chair used in seated imaging. In a case where the subject angle is determined by imaging with a camera, the subject angle may be determined by analyzing an orientation of the subject, an installation position of the pole, and/or an installation orientation of the chair, from the captured image, through image recognition or the like. Furthermore, the subject angle may be determined by detecting a physical amount relating to the subject, the pole and/or the chair, by using a physical sensor. The subject angle may be determined by recognizing the orientation of the subject by calculating a body thickness of the subject from the X-ray dose of the projection data acquired with the imaging control function 411. The body thickness of the subject may be, for example, projection data acquired by a positioning scan and/or a main scan. In a case where the projection data acquired by the main scan is used, step S11 and step S12 may be performed after step S13.

As described above, according to the first modification, it is possible to mechanically and accurately determine the subject angle.

### Second Modification

The X-ray computed tomography apparatus according to the first embodiment performs correction by directly rewriting projection data. An X-ray computed tomography apparatus according to the second modification performs correction by rewriting projection data read out to a RAM at the time of reconstruction. The second modification relates to a method of correcting the detection angle information in step S14 of the first embodiment, and step S14 in FIG. 5 will be described. It is assumed that the memory 42 provided in the console 40 stores the projection data and the detection angle information of the projection image before step S14.

In step S14, the processing circuitry 41 corrects the detection angle information of the projection data read from the memory 42 for pre-processing of reconstruction. Specifically, the detection angle information of the projection data read from the memory 42 to the RAM or the like in the DAS 33 at the time of the reconstruction pre-processing is corrected based on the correction amount. The projection data to be corrected is projection data loaded into the RAM. The projection data stored in the memory 42 may be stored without being corrected.

As described above, according to the second modification, it is possible to correct the orientation of the subject on the CT image without directly rewriting the projection data.

### Third Modification

The X-ray computed tomography apparatus according to the first embodiment reconstructs the CT image based on the projection data and the corrected detection angle information after CT imaging. An X-ray computed tomography apparatus according to the third modification reconstructs a real-time CT image based on projection data of a view range counted back from the current view number by the number of views necessary for image reconstruction and the corrected detection angle information at the time of CT imaging.

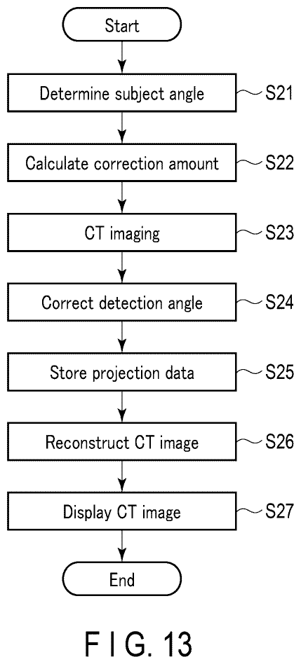

The real-time CT image is displayed without post-processing of reconstruction. As shown in FIG. 5 or FIG. 13, the correction of the projection data in the first embodiment is all performed as pre-processing of reconstruction. Therefore, the present embodiment is applicable to a real-time CT image as well. Furthermore, through implementation of the display control function 415, the processing circuitry 51 displays real-time CT images one by one on the display 43. The X-ray computed tomography apparatus according to the third modification is capable of correcting and displaying the orientation of the subject for a positioning image as well.

As described above, according to the third modification, it is possible to display a real-time CT image in which the orientation of the subject has been corrected. The third modification is applicable to the second modification as well.

### Fourth Modification

The X-ray computed tomography apparatus according to the first embodiment corrects the orientation of the subject on the CT image for a single CT imaging operation. An X-ray computed tomography apparatus according to the fourth modification performs correction to align orientations of the subject on CT images for a plurality of CT imaging operations.

The DAS 33 calculates, through implementation of the correction function 322, a correction amount relating to second CT imaging in such a manner that a positional relationship between a reference angle and a subject angle in first CT imaging by the gantry 10 in which the central axis A1 of the bore 15 is directed toward the Y-axis direction or the Z-axis direction substantially coincides with a positional relationship between a reference angle and a subject angle in the second CT imaging by the gantry 10 in which the central axis A1 of the bore 15 is directed toward a direction different from that in the first CT imaging. The first CT imaging is, as an example, decubitus imaging, that is, imaging in which the central axis A1 of the bore 15 is directed toward the Z-axis direction. The second CT imaging is, as an example, upright imaging, that is, imaging in which the central axis A1 of the bore 15 is directed toward the Y-axis direction.

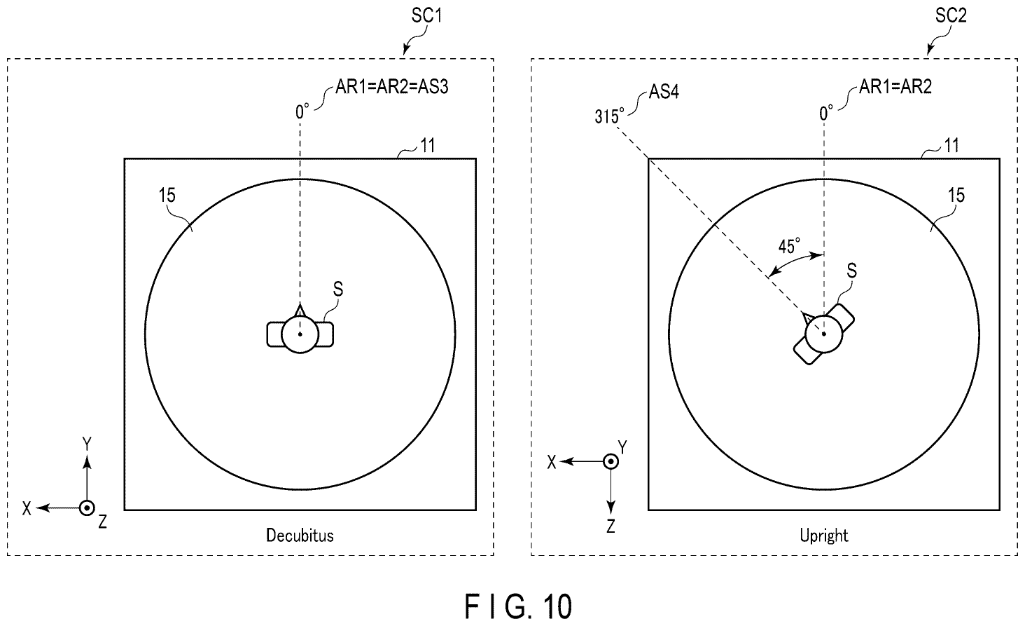

FIG. 10 is a diagram showing an orientation of the subject S in the bore 15 in decubitus imaging SC1 and upright imaging SC2. The left diagram shows the decubitus imaging SC1. At this time, the gantry body 11 and the pillar 13 take the postures shown in FIG. 3. The right diagram shows the upright imaging SC2. At this time, the gantry body 11 and the pillar 13 take the posture shown in FIG. 2. In the decubitus imaging SC1, the first reference angle AR1, the second reference angle AR2, and the subject angle AS3 coincide with each other at 0°. If reconstruction is performed based on the projection data acquired in this state, a CT image in which the front direction of the subject S is directed toward approximately the center of the upper side is obtained. On the other hand, in the upright imaging SC2, the first reference angle AR1 and the second reference angle AR2 coincide with each other at 0°, but the subject angle AS4 is 315°. If reconstruction is performed based on the uncorrected projection data acquired in this state, a CT image in which the front direction of the subject S is directed toward the direction +45° from approximately the center of the upper side in the rotating reference frame of the CT image is obtained.

To match the orientation of the subject in the projection data acquired by the upright imaging SC2 with the orientation of the subject in the projection data acquired by the decubitus imaging SC1, the processing circuitry 41 performs correction in such a manner that the detection angle information of the subject angle AS4 in the upright imaging SC2 coincides with the second subject angle. Specifically, as the correction, the processing circuitry 41 performs the correction described in the first embodiment, the second embodiment, or the second modification. The upright imaging SC2 may be the first CT imaging, and the decubitus imaging SC1 may be the second CT imaging. In this case, in FIG. 10, the detection angle information of the subject angle AS3 in the left diagram may be corrected so as to coincide with the subject angle AS4 in the right diagram. The number of pieces of projection data to be corrected may be three or more. In this case, the first CT imaging may be fixed, and new CT imaging may be set as the second CT imaging.

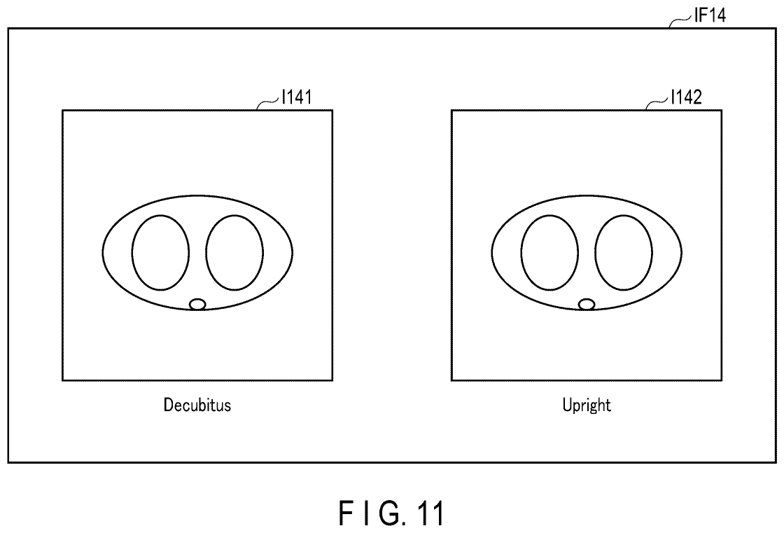

FIG. 11 is a diagram showing an example of a display screen IF14 including a CT image I141 based on the uncorrected projection data acquired in the decubitus imaging SC1 and a CT image I142 based on the corrected projection data acquired in upright imaging SC2. On the display screen IF14, the CT images I141 and I142 in which the orientations of the subject on the CT images are aligned are displayed together. Furthermore, the processing circuitry 41 displays, on the display 43, through implementation of the display control function 415, the CT image I141 acquired by the decubitus imaging SC1 and the CT image I142 acquired by the upright imaging SC2 with visual information with which the respective CT images can be identified. As the visual information, for example, text indicating a body posture such as “decubitus” and “upright” may be displayed.

The number of CT images displayed together may be three or more. The visual information to be displayed is not limited to the text indicating the body posture. For example, it may be a diagram representing a body posture, or identification can be made by color.

As described above, according to the fourth modification, it is possible to display a plurality of CT images unified in the orientation of the subject of one piece of projection data from the projection data obtained by imaging the subject directed toward the directions of different angles in the bore. Moreover, it is possible to identify each CT image by displaying visual information together with each CT image.

### Second Embodiment

In the X-ray computed tomography apparatus according to the first embodiment, the correction function 332 is included in the gantry 10. In an X-ray computed tomography apparatus according to the second embodiment, the correction function is included in the console 50. The X-ray computed tomography apparatus according to the second embodiment will be described below. Structural elements having the same functions as those included in the first embodiment will be denoted by the same reference symbols, and the same explanation will be given only where necessary.

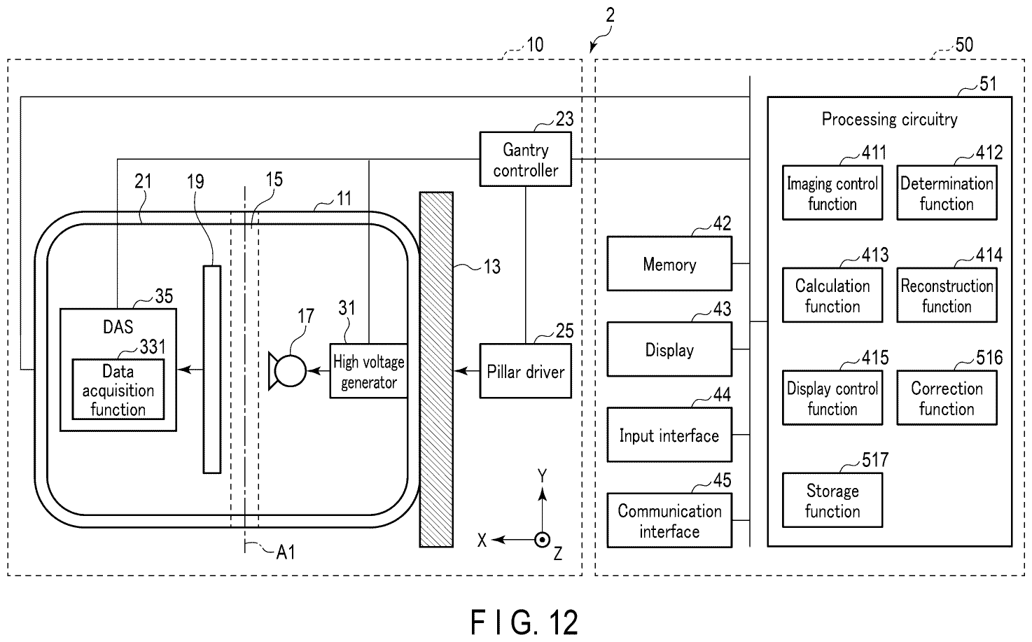

FIG. 12 is a diagram showing a configuration of an X-ray computed tomography apparatus 2 according to the second embodiment. As shown in FIG. 12, the X-ray computed tomography apparatus 2 accommodates the DAS 35 in the gantry 10. The DAS 35 acquires projection data with the data acquisition function 331.

Processing circuitry 51 executes, with the processor that executes the program loaded into the memory, an imaging control function 411, a determination function 412, a calculation function 413, a reconstruction function 414, a display control function 415, a correction function 516, a storage function 517, and the like. The functions 411 to 415, 516 and 517 are not limited to those realized by single processing circuitry. Processing circuitry may be configured by combining a plurality of independent processors that execute respective programs to implement the respective functions 411 to 415, 516 and 517. The correction function 516 corresponds to the correction function 332.

As the correction function 516, the processing circuitry 51 corrects the detection angle information of the projection data in accordance with the correction amount based on the first reference angle and the subject angle.

As the storage function 517, the processing circuitry 51 stores the projection data in the memory 52. For example, the processing circuitry 51 may store, in the memory 52, projection data in which the detection angle information is corrected. The correction amount may be stored in association with the projection data.

An operation example of the X-ray computed tomography apparatus according to the second embodiment will be described below.

FIG. 13 shows a processing procedure of a CT examination according to the second embodiment. As shown in FIG. 13, the processing circuitry 51 determines a subject angle through implementation of the determination function 512 (step S21). Step S21 corresponds to step S11 of the first embodiment.

Upon performing step S21, the processing circuitry 51 calculates, through implementation of the calculation function 513, a correction amount in such a manner that detection angle information corresponding to the subject angle determined in step S21 substantially coincides with the second reference angle (step S22). Step S22 corresponds to step S12 of the first embodiment.

Upon performing step S22, the processing circuitry 51 performs CT imaging through implementation of the imaging control function 511 (step S23). In step S23, the DAS 35 acquires projection data. The non-contact data transmission device transmits the projection data acquired by the DAS 35 to the console 50.

Upon performing step S23, the processing circuitry 51 corrects, through implementation of the correction function 516, the detection angle information of the projection data acquired in step S23 in accordance with the correction amount calculated in step S22 before the projection data is stored in the memory 52 (step S24).

Upon performing step S24, the processing circuitry 51 stores, in the memory 52, through implementation of the storage function 516, the projection data in which the detection angle information has been corrected (step S25).

Upon performing step S25, the processing circuitry 51 reconstructs the CT image through implementation of the reconstruction function 514 (step S26). Step S26 corresponds to step S15 of the first embodiment.

Upon performing step S26, the processing circuitry 51 displays, on the display 53, through implementation of the display control function 515, the CT image reconstructed in step S26 (step S27). Step S27 corresponds to step S16 of the first embodiment.

Upon performing step S27, the CT examination according to the second embodiment ends.

The correction of the orientation of the subject on the CT image shown in FIG. 13 is an example, and various additions, changes, and/or deletions can be made without changing the gist of the second embodiment.

The first to fourth modifications can also be applied to the second embodiment, and in the second modification, the processing circuitry 51 may store, in the memory 51, through implementation of the storage function 517, the correction amount calculated in step S22 in association with the projection data. In this case, the processing circuitry 51 corrects, through implementation of the correction function 516, the detection angle information in accordance with the correction amount stored in step S25.

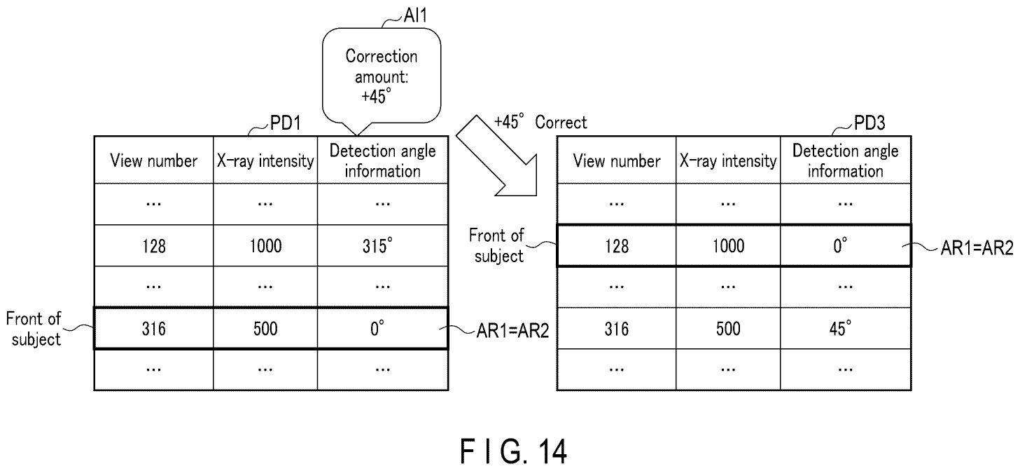

FIG. 14 is a diagram showing an example of correction based on the correction amount stored in association with the projection data as supplementary information. FIG. 14 shows uncorrected projection data PD1, a correction amount AI1 stored in association with the uncorrected projection data PD1, and corrected projection data PD3 loaded into the RAM. The uncorrected projection data PD1 corresponds to the uncorrected projection data in FIGS. 8 and 9. The correction amount AI1 is a correction amount calculated in step S22, which is stored in step S25 in the second embodiment. The correction amount is, for example, +45°. The processing circuitry 51 corrects, based on the correction amount AI1, the uncorrected projection data PD1 read to the RAM of the processing circuitry 51 at the time of reconstruction, and generates the corrected projection data PD3. By storing the correction amount in association with the projection data, it is possible to correct the orientation of the subject on the CT image even after the end of the CT examination.

### In Sum

The X-ray computed tomography apparatus according to the present embodiment includes the gantry body 11 and the processing circuitry 51. The gantry body 11 supports the X-ray tube 17 and the X-ray detector 19 to be rotatable about the central axis A1 of the bore 15. The processing circuitry 51 corrects the detection angle information of the projection data in accordance with the correction amount based on the first reference angle in the rotation direction of the X-ray tube 17 and the subject angle that determines the orientation of the subject in the bore 15, and reconstructs a CT image relating to the subject based on the projection data and the corrected detection angle information.

Herein, the present embodiment is compared with a comparative example in which the orientation of the subject on the CT image is corrected as post-processing of reconstruction of the CT image. In the comparative example, the orientation of the subject on the CT image is corrected by performing CT image rotation processing as post-processing on the reconstructed CT image. However, if the rotation angle is not an integral multiple of 90°, the pixel value before the rotation does not correspond to the pixel value after the rotation on a one-to-one basis, and thus blurring of the image occurs in the pixel. Furthermore, because rotation processing is performed as post-processing of reconstruction, it takes a lot of time to check the corrected CT image. Therefore, a workflow from imaging to interpretation for the CT image not requiring correction is hindered. As compared with the comparative example, according to the present embodiment, the detection angle information of the projection data is corrected as pre-processing of reconstruction. Therefore, because the detection angle information is corrected, blurring of the image as in the comparative example does not occur, and the image quality can be improved. Furthermore, since it is only necessary to correct the detection angle information, the reconstruction processing is the same as that of the comparative example and can be easily implemented. Moreover, by performing the correction, the subject image can be directed toward a direction desired by the user.

### Third Embodiment

The X-ray computed tomography apparatuses according to the first and second embodiments correct the detection angle information for correcting the orientation of the subject on the CT image. The X-ray computed tomography apparatus according to the third embodiment corrects an exposure start angle. The exposure start angle is an angle in the rotation direction of the X-ray tube 17 at which the X-ray tube 17 starts exposure of X-rays in CT imaging. Processing circuitry 61 of the third embodiment may correct the exposure start angle in such a manner that the second CT imaging is performed on the subject with the same X-ray tube 17 trajectory as that of the first CT imaging. A console 60 corresponds to the console 40 of the first embodiment.

An X-ray computed tomography apparatus according to the third embodiment will be described below. Structural elements having the same operations as those in the first or second embodiment will be denoted by the same reference symbols, and the same explanation will be given only where necessary.

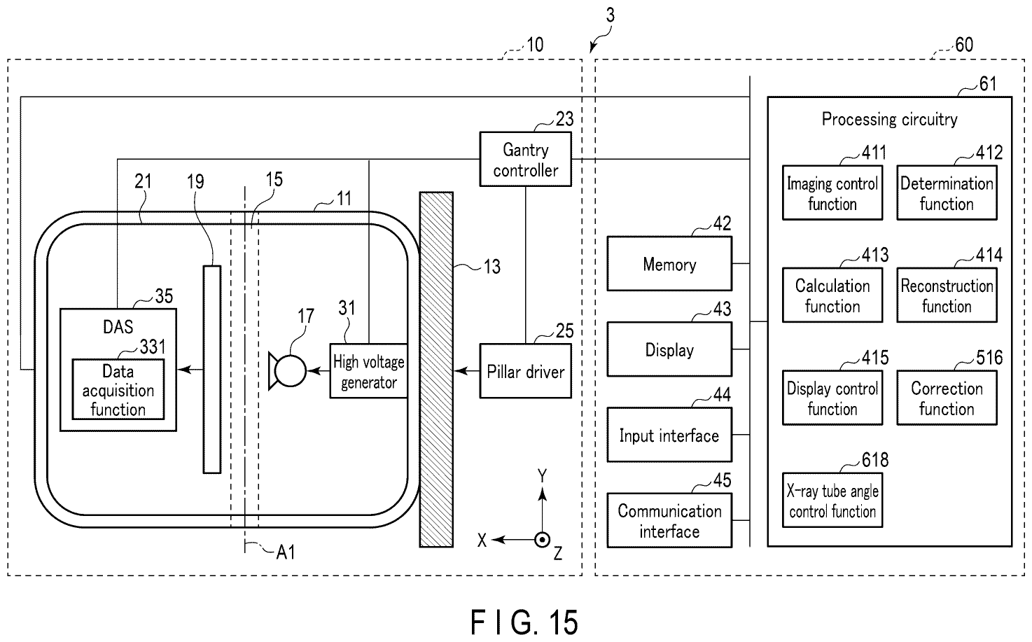

FIG. 15 is a diagram showing a configuration of an X-ray computed tomography apparatus 3 according to the third embodiment. As shown in FIG. 15, the processing circuitry 61 executes, using the processor that executes the program loaded into the memory, an imaging control function 411, a determination function 412, a calculation function 413, a reconstruction function 414, a display control function 415, a correction function 516, an X-ray tube angle control function 618, and the like. The functions 411 to 415, 516, and 618 are not limited to those realized by single processing circuitry.

Processing circuitry may be configured by combining a plurality of independent processors that execute respective programs to implement the respective functions 411 to 415, 516 and 618.

As the calculation function 413, the processing circuitry 61 calculates a correction amount of a second exposure start angle of the X-ray tube 17 based on a first exposure start angle of the X-ray tube 17, a current position of the X-ray tube 17, and the subject angle. The first exposure start angle is an exposure start angle in the first CT imaging. The second exposure start angle is an exposure start angle in the second CT imaging. For example, the exposure start angle is controlled at the first reference angle.

As the correction function 516, the processing circuitry 61 corrects the second exposure start angle based on the calculated correction amount before the second CT imaging.

As the X-ray tube angle control function 618, the processing circuitry 61 moves the X-ray tube 17 to the second exposure start angle corrected with the correction function 516. Specifically, the X-ray tube 17 is moved from the current position by the correction amount about the central axis Al of the bore 15, and the angle of the X-ray tube 17 after the movement is set as the second exposure start angle.

An operation example of the X-ray computed tomography apparatus according to the third embodiment will be described below.

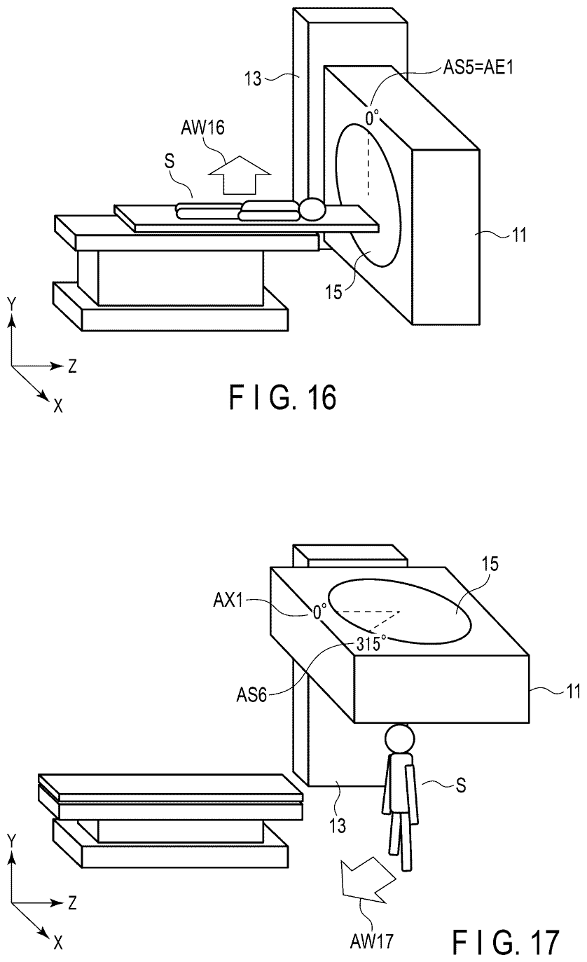

FIG. 16 is a view showing a first exposure start angle AE1 in the first CT imaging. As shown in FIG. 16, the first CT imaging is, for example, decubitus imaging. In FIG. 16, the first exposure start angle AE1 is 0°. An orientation AW16 of the subject S is the front direction of the subject. A subject angle AS5 is 0°. In this case, the X-ray tube 17 starts exposure from the front direction of the subject S.

FIG. 17 is a view showing an uncorrected angle AX1 at which the X-ray tube 17 is positioned in the second CT imaging. As shown in FIG. 17, the second CT imaging is, for example, upright imaging. In FIG. 17, the angle AX1 is 0°. An orientation AW17 of the subject S is the front direction of the subject. The subject angle is 315°. In this case, the X-ray tube 17 starts exposure from a direction shifted by 45° from the front direction of the subject S.

In order for the X-ray tube 17 to take the same trajectory with respect to the subject S in the first CT imaging and the second CT imaging, the processing circuitry 61 calculates, through implementation of the calculation function 413, a correction amount for matching the first exposure start angle AE1 and the second exposure start angle with respect to the subject angle. As an example, the correction amount is calculated by subtracting the subject angle from the angle AX1 or an angle obtained by adding 360° to the angle AX1. Specifically, in FIG. 17, the correction amount of the second exposure start angle may be calculated to be +315° or −45°. The processing circuitry 61 corrects, through implementation of the correction function 516, the second exposure start angle in accordance with the calculated correction amount. Specifically, in FIG. 17, the second exposure start angle may be corrected to be +315° or −45° from the angle AX1 based on the calculated correction amount.

The processing circuitry 61 moves, through implementation of the X-ray tube angle control function 618, the exposure start angle of the X-ray tube 17 in the second CT imaging in accordance with the calculated correction amount. In FIG. 17, the second exposure start angle is moved by +315° or −45° from 0° of the angle AX1 to be an angle of 315°. Thus, in the first CT imaging and the second CT imaging of different body positions, it is possible to match the trajectory of the X-ray tube 17 with respect to the orientation of the subject.

### In Sum

The X-ray computed tomography apparatus according to the present embodiment includes the gantry body 11 and the processing circuitry 61. The gantry body 11 supports the X-ray tube 17 and the X-ray detector 19 to be rotatable about the central axis Al of the bore 15. The processing circuitry 61 corrects the second exposure start angle in accordance with a correction amount based on the first exposure start angle in the rotation direction of the X-ray tube 17 and the subject angle.

Herein, the present embodiment will be compared with a comparative example in which the first CT imaging and the second CT imaging are performed without matching the first exposure start angle and the second exposure start angle with respect to the orientation of the subject. In the comparative example, in the first CT imaging and the second CT imaging, exposure of X-rays is started from different exposure start angles with respect to the orientation of the subject. In the CT image, noise derived from the trajectory of the X-ray tube appears. Therefore, in the comparative example, noise derived from the trajectory of the X-ray tube appears in different directions in the CT images acquired by the first CT imaging and the second CT imaging, resulting in difficulty in comparing the CT images. As compared with the comparative example, according to the present embodiment, the first CT imaging and the second CT imaging are performed by matching the first exposure start angle and the second exposure start angle with respect to the orientation of the subject. Therefore, by making the tendency of noise similar between the CT images to be reconstructed, distinction between noise and the subject image can be facilitated.

According to at least one of the embodiments described above, it is possible to generate a CT image that the user can easily check.

The term “processor” used herein refers to, for example, a CPU or a GPU, or various types of circuitry, such as an application-specific integrated circuit (ASIC), a programmable logic device (e.g., a simple programmable logic device (SPLD), a complex programmable logic device (CPLD), or a field programmable gate array (FPGA)), and so on. The processor realizes functions by reading and executing a program stored in the memory circuitry. The program may be directly incorporated into the circuit of the processor instead of being stored in the storage circuit. In this case, the processor implements the function by reading and executing the program incorporated into the circuit. On the other hand, if the processor is an ASIC, for example, the functions are directly incorporated into the circuitry of the processor as logic circuits, instead of the programs being stored in the storage circuitry. The processors described in connection with the above embodiment are not limited to single-circuit processors; a plurality of independent circuits may be integrated into a single processor that realizes the functions. Moreover, the structural elements in FIG. 1, FIG. 12 and FIG. 15 may be integrated into one processor, and the processor may implement the functions of the structural elements.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An X-ray computed tomography apparatus comprising:
a gantry body configured to support an X-ray tube, an X-ray detector, and data acquisition circuitry to be rotatable about a central axis of a bore, the X-ray tube generating X-rays, the X-ray detector detecting X-rays generated by the X-ray tube and passing through a subject, the data acquisition circuitry acquiring projection data via the X-ray detector; and
processor configured to correct detection angle information of the projection data in accordance with a correction amount based on a first reference angle in a rotation direction of the X-ray tube and a subject angle determining an orientation of the subject in the bore, and reconstruct a CT image relating to the subject based on the projection data and the corrected detection angle information.

2. The X-ray computed tomography apparatus according to claim 1, wherein the processor is further configured to reconstruct the CT image in such a manner that the subject angle coincides with a second reference angle in the CT image.

3. The X-ray computed tomography apparatus according to claim 2, wherein the second reference angle is an angle at which a rotation angle about a center point of the CT image is positioned at approximately a center of an upper side.

4. The X-ray computed tomography apparatus according to claim 1, wherein the processor is provided in the gantry body and configured to correct the detection angle information in accordance with the correction amount at the time of acquiring the projection data.

5. The X-ray computed tomography apparatus according to claim 1, further comprising:
a memory provided in a console and configured to store the projection data,
wherein the processor is provided in the console and configured to correct the detection angle information before the projection data is stored in the memory, and
the memory is configured to store the projection data in which the detection angle information is corrected.

6. The X-ray computed tomography apparatus according to claim 1, further comprising:
a memory provided in a console and configured to store the projection data,
wherein the processor is configured to correct the detection angle information of the projection data read from the memory for pre-processing of reconstruction.

7. The X-ray computed tomography apparatus according to claim 6, wherein
the memory is configured to store the correction amount in association with the projection data, and
the processor is configured to correct the detection angle information in accordance with the correction amount stored in association with the projection data.

8. The X-ray computed tomography apparatus according to claim 4, wherein
the processor is configured to reconstruct, at a time of CT imaging, real-time CT images based on the projection data of a view range counted back by a number of views required for image reconstruction from a current view number and the corrected detection angle information, and display the real-time CT images one by one on a display device.

9. The X-ray computed tomography apparatus according to claim 5, wherein
the processor is configured to reconstruct, at a time of CT imaging, real-time CT images based on the projection data of a view range counted back by a number of views required for image reconstruction from a current view number and the corrected detection angle information, and display the real-time CT images one by one on a display device.

10. The X-ray computed tomography apparatus according to claim 6, wherein
the processor is configured to reconstruct, at a time of CT imaging, real-time CT images based on the projection data of a view range counted back by a number of views required for image reconstruction from a current view number and the corrected detection angle information, and display the real-time CT images one by one on a display device.

11. The X-ray computed tomography apparatus according to claim 1. wherein the subject angle is an angle in a front direction of the subject.

12. The X-ray computed tomography apparatus according to claim 1, wherein the processor is configured to determine the subject angle according to an instruction of a user.

13. The X-ray computed tomography apparatus according to claim 1, wherein the processor is configured to determine the subject angle based on the projection data.

14. The X-ray computed tomography apparatus according to claim 1, further comprising:
a camera configured to generate an optical image,
wherein the processor is configured to determine the subject angle based on the optical image generated by the camera.

15. The X-ray computed tomography apparatus according to claim 1, further comprising:
a physical sensor capable of detecting a physical amount,
wherein the processor is configured to determine the subject angle based on the physical amount detected by the physical sensor.

16. The X-ray computed tomography apparatus according to claim 1, further comprising:
a pillar configured to support the gantry body to be rotatable about a horizontal axis in such a manner that the bore is directed toward a horizontal direction or a perpendicular direction with respect to a floor surface,
wherein the processor is configured to:
calculate the correction amount relating to second CT imaging in such a manner that a positional relation between the first reference angle and the detection angle information corresponding to the subject angle in first CT imaging by the gantry body in which the bore is directed toward the horizontal direction or the perpendicular direction with respect to the floor surface approximately coincides with a positional relationship between the first reference angle and the detection angle information corresponding to the subject angle in the second CT imaging by the gantry body in which the bore is directed toward a direction different from the direction of the first CT imaging;
correct the detection angle information relating to the second CT imaging in accordance with the correction amount;
reconstruct a CT image based on the projection data and the corrected detection angle information; and
display a CT image acquired by the first CT imaging with a CT image acquired by the second CT imaging on a display device.

17. The X-ray computed tomography apparatus according to claim 16, wherein the processor is configured to display the CT image acquired by the first CT imaging and the CT image acquired by the second imaging with visual information that allows identification of the respective CT images on the display device.

18. The X-ray computed tomography apparatus according to claim 1, wherein the processor is configured to correct an exposure start angle at which the X-ray tube starts exposure of X-rays in CT imaging in accordance with the correction amount in the rotation direction of the X-ray tube.

19. An X-ray computed tomography apparatus comprising:
a gantry body configured to support an X-ray tube, an X-ray detector, and data acquisition circuitry to be rotatable about a central axis of a bore, the X-ray tube generating X-rays, the X-ray detector detecting X-rays generated by the X-ray tube and passing through a subject, the data acquisition circuitry acquiring projection data via the X-ray detector; and
processor configured to correct an exposure start angle at which the X-ray tube starts exposure of X-rays in CT imaging in accordance with a correction amount based on a first reference angle in a rotation direction of the X-ray tube and a subject angle determining an orientation of the subject in the bore.
